(19) United States
(12) Patent Application Publication
(10) Pub. No.: US 2005/0175980 A1
(43) Pub. Date: Aug. 11, 2005

(54) Quantitative assay of the angiogenic and antiangiogenic activity of a test molecule

(75) Inventors: Steven K. Libutti, North Potomac, MD (US); Mark L. Kayton, New York, NY (US)

Correspondence Address: NATIONAL INSTITUTE OF HEALTH;C/O NEEDLE & ROSENBERG, P.C., SUITE 1000, 999 PEACHTREE STREET, ATLANTA, GA, 30303, US

(73) Assignee: Government of the United States of America, repres by the Secretary, Dept. of Health and Human Serv., Rockville, MD (US)

(21) Appl. No.: 10/510,652

(22) PCT Filed: Apr. 9, 2003
(86) PCT No.: PCT/US03/10932

Related U.S. Application Data
(60) Provisional application No. 60/371,010, filed on Apr. 9, 2002.

(51) Int. Cl. C12Q 1/00

(52) U.S. Cl. 435/4

(57) ABSTRACT

A method of measuring the angiogenic or antiangiogenic activity of a test molecule comprising (i) utilizing a modified CAM assay to assign an FVD value for a test region of interest and comparing this value to an FVD value for a control region of interest, or (ii) utilizing a modified CAM assay to determine the spectrophotometric absorbance value of a test region of interest and comparing this value to the spectrophotometric absorbance value of a control region of interest, wherein a lower value of the test region of interest as compared to the of the control region of interest is indicative of the test molecule being useful as an inhibitor of angiogenesis, and wherein a higher value of the test region of interest as compared to the value of the control region of interest is indicative of the test molecule being useful as a stimulator of angiogenesis.

### FIELD OF THE INVENTION

This invention pertains to a method of measuring the angiogenic or antiangiogenic activity of a test molecule.

### BACKGROUND OF THE INVENTION

Angiogenesis can be defined as the process by which new blood vessels form from pre-existing vessels. This biological process is very complex and must be highly regulated to prevent certain disease states from occurring. Indeed, when vascularization is not stringently controlled, serious pathologies can result. For example, reduced vascularization of target tissues is associated with ischemic damage, vascular diseases, necrosis, and muscle wasting. Increased vascularization is associated with, for instance, tumor growth, edema, and diseases of the eye, such as diabetic retinopathy and the exudative form of age-related macular degeneration, which are major causes of blindness worldwide.

Because angiogenesis is a highly complex biological process that involves a number of different molecules and signaling pathways, the development of various strategies to study this process has been an area of concentration for many research groups. To date, both in vitro and in vivo strategies have been established to identify putative compounds which either stimulate or inhibit angiogenesis. For example, some in vitro strategies include the Matrigel tube-forming assay (see, e.g., Grant et al., Cell, 58:933-943 (1989); and Davis et al., J. Cell. Biochem., 51:206-218 (1993)), the fibrin and collagen gel-cord-forming assays (see, e.g., Dvorak et al., Lab. Invest., 57:673-686 (1987); and Montesano et al., J. Cell. Biol., 97:1648-1652 (1983)), the aortic ring model (see, e.g., Brown et al., Lab. Invest., 75:539-555 (1996)), and a variety of endothelial cell proliferation assays (see, e.g., Frater-Schroder et al., PNAS, 84:5277-5281 (1987)). These strategies are limited, however, in that they can provide only limited information of the angiogenic cascade as a whole. Accordingly, various in vivo strategies have been developed to analyze more thoroughly the process of angiogenesis. Some of these in vivo strategies include the rat, mouse and rabbit corneal pocket assays (see, e.g., Sholley et al., Lab. Invest., 51:624-634 (1994); Friedlander et al., Science, 270:1500-1502 (1995); and Chen et al., Cancer Res., 55:4230-4233 (1995)), the primate iris neovascularization model (see, e.g., Miller et al., Am. J. Pathol., 145:574-584 (1994)), the human/mouse chimeric angiogenesis assay (see, e.g., Brooks et al., J. Clin. Invest., 96:1815-1822 (1995)), a murine Matrigel plug assay (see, e.g., Passaniti et al., Lab. Invest., 67:519-527 (1992)), and the chick embryo chorioallantoic membrane (CAM) assay (see, e.g., Brooks et al., Science, 264:569-571 (1994); and Nguyen et al., Microvasc. Res., 47:31-40 (1994)). These strategies are limited, however, by the length of time required for the assay and the complexity and expense to carry them out. Furthermore, most of the physiological effects of these molecules can generally only be monitored locally, with little information available as to their systemic effects.

Recently, a strategy has been developed which involves modifying the CAM assay. The CAM is the major respiratory structure for the exchange of gases and nutrients during embryonic development and, thus, becomes highly vascularized. In this regard, the CAM provides an ideal microenvironment in which to study the angiogenic or antiangiogenic activity of a test molecule. The modified CAM assay developed by Brooks et al. (see Brooks et al., “Use of the 10-Day-Old Chick Embryo Model for Studying Angiogenesis,” in Methods in Molecular Biology, Vol.129: Integrin Protocols, ed. A. R. Howlett (Totowa, N.J.: Humana Press, 1999)) involves placing a filter disc containing angiogenic cytokines on the CAM. Following induction of angiogenesis, various inhibitors of angiogenesis can be administered either intravenously or topically and evaluated for their effects. Since new blood vessel development is initiated from pre-existing vessels, the effects of the inhibitor can be evaluated. However, this method quantifies the angiogenic response with the use of a stereomicroscope by counting the number of blood vessel branch points within the CAM directly beneath the filter disc. Such a quantification method is very time-consuming and may not provide accurate results.

In view of the above, there remains a need to develop a strategy of measuring the angiogenic or antiangiogenic activity of a test molecule which is easy to perform, which is cost-effective, and which gives accurate results (i.e., is easy to quantify an angiogenic effect). The invention provides such a method. This and other objects and advantages of the present invention, as well as additional inventive features, will be apparent from the description of the invention provided herein.

### BRIEF SUMMARY OF THE INVENTION

The invention provides a method of measuring the angiogenic or antiangiogenic activity of a test molecule. In one embodiment, the method comprises obtaining an embryonated fowl egg, creating a window in the shell of the fowl egg, such that the CAM membrane is exposed, providing to a test region of interest on the CAM a substrate, administering to a vessel located in the CAM a test molecule, administering to a vessel located in the CAM a fluorescent-labeled particle, such that the fluorescent-labeled particle travels through each vessel contained in the test region of interest, removing the substrate and the test region of interest from the fowl egg, capturing a three-dimensional image of the test region of interest, wherein the three-dimensional image comprises a plurality of pixels, such that a fluorescent vascular density (FVD) value can be assigned to the test region of interest, and comparing the FVD value of the test region of interest with the FVD value of a control region of interest that was prepared in the same manner as the test region of interest but without the administration of a test molecule or with the administration of a control molecule, such that the angiogenic or antiangiogenic activity of the test molecule is measured. A lower FVD value of the test region of interest as compared to the FVD value of the control region of interest is indicative of the test molecule being useful as an inhibitor of angiogenesis. Conversely, a higher FVD value of the test region of interest as compared to the FVD value of the control region of interest is indicative of the test molecule being useful as a stimulator of angiogenesis.

In another embodiment, the method comprises obtaining an embryonated fowl egg, creating a window in the shell of the fowl egg, such that the CAM is exposed, providing to a test region of interest on the CAM a substrate, administering to a vessel located in the CAM a test molecule, administering to a vessel located in the CAM an agent to measure metabolic activity, removing the substrate and the test region of interest from the fowl egg, measuring the spectrophotometric absorbance value of the test region of interest, and comparing the spectrophotometric absorbance value of the test region of interest with the spectrophotometric absorbance value of a control region of interest that was prepared in the same manner as the test region of interest but without the administration of a test molecule or with the administration of a control molecule, such that the angiogenic or antiangiogenic activity of the test molecule is measured. A lower spectrophotometric absorbance value of the test region of interest as compared to the spectrophotometric absorbance value of the control region of interest is indicative of the test molecule being useful as an inhibitor of angiogenesis. Conversely, a higher spectrophotometric absorbance value of the test region of interest as compared to the spectrophotometric absorbance value of the control region of interest is indicative of the test molecule being useful as a stimulator of angiogenesis.

### DETAILED DESCRIPTION OF THE INVENTION

The present invention is directed to a modified version of the CAM assay to measure the angiogenic or antiangiogenic activity of a test molecule. In a typical CAM assay, a 10-day old embryonated chick embryo is obtained. A filter disc containing a stimulator or inhibitor of angiogenesis is then placed on the CAM of the embryo to stimulate or inhibit angiogenesis in this area. A test molecule is then administered and evaluated for its effects on the stimulation or inhibition of blood vessel growth in the area associated with the filter disc. This angiogenic response can then be quantified with the use of a microscope, such as a light microscope or a stereomicroscope, by counting the number of blood vessel branch points within the CAM directly beneath the filter disc. Because such a typical CAM assay is limited by complexity and accuracy, the present inventive method modifies the assay to involve the administration of a fluorescent-labeled particle to the CAM before the angiogenic or antiangiogenic activity is measured, such that the angiogenic or antiangiogenic activity of a test molecule can be measured by more effective means (e.g., by laser confocal microscopy). Moreover, by performing the complete assay in the shell prior to imaging, the embryo can be kept alive virtually until hatching (i.e., day 21-22 of gestation), which allows one to study vessel development when most of the CAM has already developed (i.e., after day 10 of gestation). Also, by performing the complete assay in the shell, the test molecule can be delivered systemically, as compared to locally, to allow for more accurate results to be obtained. By employing the modifications outlined above, a more efficient and accurate means of measuring angiogenic or antiangiogenic activity of a test molecule is achieved.

In view of the above, the present invention provides a method of measuring the angiogenic or antiangiogenic activity of a test molecule. In one embodiment, the method comprises (a) obtaining an embryonated fowl egg, (b) creating a window in the shell of the fowl egg, such that the CAM is exposed, (c) providing to a test region of interest on the CAM a substrate, (d) administering to a vessel located in the CAM a test molecule, (e) administering to a vessel located in the CAM a fluorescent-labeled particle, such that the fluorescent-labeled particle travels through each vessel contained in the test region of interest, (f) removing the substrate and the test region of interest from the fowl egg, (g) capturing a three-dimensional image of the test region of interest, wherein the three-dimensional image comprises a plurality of pixels, such that a fluorescent vascular density (FVD) value can be assigned to the test region of interest, and (h) comparing the FVD value of the test region of interest with the FVD value of a control region of interest that was prepared in the same manner as the test region of interest but without the administration of a test molecule or with the administration of a control molecule, such that the angiogenic or antiangiogenic activity of the test molecule is measured. A lower FVD value of the test region of interest as compared to the FVD value of the control region of interest is indicative of the test molecule being useful as an inhibitor of angiogenesis. Conversely, a higher FVD value of the test region of interest as compared to the FVD value of the control region of interest is indicative of the test molecule being useful as a stimulator of angiogenesis.

In another embodiment, the method comprises comprises (a) obtaining an embryonated fowl egg, (b) creating a window in the shell of the fowl egg, such that the CAM is exposed, (c) providing to a test region of interest on the CAM a substrate, (d) administering to a vessel located in the CAM a test molecule, (e) administering to a vessel located in the CAM an agent to measure metabolic activity, (f) removing the substrate and the test region of interest from the fowl egg, (g) measuring the spectrophotometric absorbance value of the test region of interest, and (h) comparing the spectrophotometric absorbance value of the test region of interest with the spectrophotometric absorbance value of a control region of interest that was prepared in the same manner as the test region of interest but without the administration of a test molecule or with the administration of a control molecule, such that the angiogenic or antiangiogenic activity of the test molecule is measured. A lower spectrophotometric absorbance value of the test region of interest as compared to the spectrophotometric absorbance value of the control region of interest is indicative of the test molecule being useful as an inhibitor of angiogenesis. Conversely, a higher spectrophotometric absorbance value of the test region of interest as compared to the spectrophotometric absorbance value of the control region of interest is indicative of the test molecule being useful as a stimulator of angiogenesis.

Any suitable “agent to measure metabolic activity” can be used in the context of the above method. Preferred agents include XTT, MTT and WST-1. The test molecule and the agent to measure metabolic activity can be administered to different vessels in the CAM. In this regard, each vessl can be cannulated prior to administration of the test molecule and the agent to measure metabolic activity.

The embryonated fowl egg used in the context of the invention can be any suitable fowl egg. Suitable fowl eggs typically include those deriving from a fowl belonging to the order Galliformes. Preferably, within the order Galliformes, the fowl egg is derived from a fowl belonging to the family Phasianidae, and, more preferably, from a fowl belonging to the genus Gallus. Most preferably, the fowl egg is derived from the fowl Gallus gallus (i.e., the chicken).

Methods of preparing a fowl egg for use in the present invention (i.e., for use in a CAM assay) are well-known in the art and are described in, for example, Brooks et al. (1994), supra, and Brooks et al. (1999), supra. It will be understood that the embryonated fowl egg can be used in a CAM assay at any stage during the formation of the embryo. Indeed, because the entire assay is performed inside the shell prior to imaging, the fowl egg can be used on any day of gestation. It is preferred, however, that the fowl egg is obtained for use at day 7-12 of gestation, and, more preferably, at day 8-11 of gestation. Most preferably, the fowl egg is obtained for use at day 10 of gestation.

The “region of interest” as described herein, refers to a region of the CAM that is later removed from the fowl egg for analysis. The region of interest will be provided with a substrate, which also is removed with the region of interest for analysis. It will be understood that the location of a region of interest will vary from fowl egg to fowl egg.

A “test region of interest” refers to a region of interest as described above that will be exposed to a test molecule, typically administered to a vessel outside of the test region of interest. The test molecule is allowed to circulate throughout the vasculature of the CAM, such that it carries out its effects in the test region of interest. A “control region of interest” refers to a region of interest that is prepared in the same manner as the test region of interest but without the administration of a test molecule or with the administration of a control molecule, typically outside of the control region of interest. Suitable control molecules typically comprise a control solution and include, for example, sodium chloride in solution, dimethylsulfoxide (DMSO), albumin, preparations of a test molecule wherein the test molecule has been inactivated (e.g., by prolonged exposure to heat), or solutions containing an antibody which renders the test molecule ineffective. By providing a control region of interest, an FVD value of the test region of interest can be compared to an FVD value of the control region of interest to analyze the angiogenic or antiangiogenic activity of the test molecule. Indeed, both experiments are carried out in the same manner but for the test molecule. Accordingly, any discrepancy between the FVD value of the test region of interest and the control region of interest is attributable to the test molecule.

The substrate for use in the context of the present invention can be any suitable substrate. For example, the substrate can be glass, plastic, nylon, silicon, polytetrafluroethylene, Matrigel, collagen, fibrinogen, agarose, methylcellulose or filter paper. Preferably, the substrate is agarose, methylcellulose or filter paper, and, most preferably, filter paper. Other suitable substrates will be recognized by those skilled in the art. Accordingly, the substrates identified herein are in no way limiting.

The substrate can further comprise a modulator of angiogenesis (i.e., angiogenesis-modulation factors). Angiogenesis-modulation factors include stimulators of angiogenesis or, in the alternative, inhibitors of angiogenesis. Thus, angiogenesis-modulation factors can influence the actual generation or inhibition of new blood vessels, as well as the quality of the blood vessels. The angiogenesis-modulation factors also can act upon different angiogenic processes. Since angiogenesis is a complex biological phenomenon that relies on several controlled angiogenic processes, any biological process involved in the stimulation or inhibition of new blood vessels, e.g., basement membrane breakdown, cell proliferation, cell migration, vessel wall maturation, lumen formation, vessel dilatation, production of mediators, branching of vessels, etc., is an “angiogenic process” that can be acted upon by an angiogenesis-modulation factor.

Preferably, the angiogenesis-modulation factor modulates angiogenic processes by acting upon a target molecule. Target molecules refer to, for example, receptors (e.g., growth factor receptors), intracellular signaling molecules, genes, gene products, such as mRNA and proteins, and chemical mediators.

In addition, as the angiogenesis-modulation factor preferably acts on a target molecule, an angiogenesis-modulation factor desirably acts upon a cellular signal transduction pathway. Different angiogenic processes rely on different effector molecules and signal transduction pathways for regulation. For example, growth factors, such as a vascular endothelial growth factor (VEGF), fibroblast growth factor (FGF), and platelet-derived growth factor (PDGF), act through a tyrosine kinase family receptor system to transmit signals to the cell nucleus. Growth factors, growth factor receptors, and their corresponding signal transduction pathways are described in the Handbook of Experimental Pharmacology, Spawn & Roberts, Eds., V95, Springer-Verlag (1990). Transcription factors, such as hypoxia-inducible factor-1alpha (HIF-1α) and PR39, do not act through tyrosine kinase receptors but, instead, act directly on DNA to promote the production of positive regulators of angiogenesis. Heparin Binding Neurotrophic Factor (HBNF) acts by up-regulating the production of urokinase, thereby promoting angiogenesis. Similarly, soluble growth factor receptors inhibit neovascularization by blocking signaling through growth factor receptors, while pigment epithelium-derived factor (PEDF) is a serine protease inhibitor that most likely influences cell-cell or cell-extracellular matrix interactions. Angiostatin works through signal transduction pathways to achieve mitotic arrest. Thus, each class of angiogenesis-modulation factor has different mechanisms of action by acting through different target molecules.

The angiogenesis-modulation factor can be used, for example, to induce or stimulate angiogenesis. By “stimulate angiogenesis” is meant that angiogenesis is either initiated or enhanced. Therefore, for example, when angiogenesis is not occurring, the angiogenesis can be initiated. However, if angiogenesis is already occurring, angiogenesis can be enhanced or heightened. When stimulation of angiogenesis is desired, the angiogenesis-modulation factor is referred to herein as a “stimulator of angiogenesis,” e.g., a factor that aids in the formation and/or quality of new blood vessels. Such factors include, but are not limited to, a synthetic molecule, a nucleic acid sequence encoding a stimulator of angiogenesis, a polypeptide that can stimulate angiogenesis, a biological tissue containing a stimulator of angiogenesis, and a cell containing a stimulator of angiogenesis. Preferably, a greater degree of angiogenesis is stimulated by the inclusion of the stimulator of angiogenesis to a substrate as compared to angiogenesis resulting from administration of the substrate alone.

Stimulators of angiogenesis are variously described in U.S. Pat. No. 5,194,596 (Tischer et al.), U.S. Pat No. 5,219,739 (Tischer et al.), U.S. Pat. No. 5,338,840 (Bayne et al.), U.S. Pat. No. 5,532,343 (Bayne et al.), U.S. Pat. No. 5,169,764 (Shooter et al.), U.S. Pat. No. 5,650,490 (Davis et al.), U.S Pat. No. 5,643,755 (Davis et al.), U.S. Pat. No. 5,879,672 (Davis et al.), U.S. Pat. No. 5,851,797 (Valenzuela et al.), U.S. Pat. No. 5,843,775 (Valenzuela et al.), and U.S. Pat. No. 5,821,124 (Valenzuela et al.); International Patent Applications WO 95/24473 (Hu et al.) and WO 98/44953 (Schaper); European Patent Documents 476 983 (Bayne et al.), 506 477 (Bayne et al.), and 550 296 (Sudo et al.); Japanese Patent Documents 1038100, 2117698, 2279698, and 3178996; J. Folkman et al., Nature, 329, 671 (1987); Fernandez et al., Circulation Research, 87, 207-213 (2000), and Moldovan et al., Circulation Research, 87, 378-384 (2000).

Alternatively, the angiogenesis-modulating factor can be used to inhibit angiogenesis. By “inhibit angiogenesis” is meant that angiogenesis is prevented or ameliorated. One of ordinary skill in the art will understand that complete prevention or amelioration (reduction) of angiogenesis is not required in order to obtain a biological effect. Therefore, the present inventive method contemplates both partial and complete prevention and amelioration of angiogenesis. When inhibition of angiogenesis is desired, the angiogenesis-modulation factor is referred to herein as an inhibitor of angiogenesis, e.g., a factor that prevents the formation and/or quality of new blood vessels. Such factors include, but are not limited to, a synthetic molecule, a nucleic acid sequence encoding an inhibitor of angiogenesis, a polypeptide that can inhibit angiogenesis, a biological tissue containing an inhibitor of angiogenesis, and a cell containing an inhibitor of angiogenesis. Preferably, a greater degree of angiogenesis is inhibited by the inclusion of the inhibitor of angiogenesis to a substrate as compared to the inhibition of angiogenesis resulting from administration of the substrate alone. Angiogenesis inhibitors also can be antagonists for angiogenesis-promoting agents, such that the angiogenesis-promoting factors are neutralized (see, e.g., Sato, PNAS, 95:5843-5844 (1998)).

More particularly, inhibitors of angiogenesis suitable for use in the present invention include, for instance, anti-angiogenic factors, cytotoxins, apoptotic factors, anti-sense molecules specific for an angiogenic factor, ribozymes specific for an angiogenic factor, receptors for an angiogenic factor, antibodies that bind to an angiogenic factor, and antibodies that bind to a receptor for an angiogenic factor. Anti-sense molecules, ribozymes, soluble receptors, and antibodies to angiogenic factors and receptors can be considered inhibitors of angiogenesis, as they sequester away positive regulators of angiogenesis.

The anti-angiogenic factors contemplated for use in the present invention include PEDF, angiostatin, thrombospondin, protamine, vasculostatin, endostatin, platelet factor 4, heparinase, interferons (e.g., INFα), and the like. One of ordinary skill in the art will appreciate that any anti-angiogenic factor can be modified or truncated and retain anti-angiogenic activity. As such, active fragments of anti-angiogenic agents (i.e., those fragments having biological activity sufficient to inhibit angiogenesis) are suitable for use in the present inventive methods. Hyaluronic acid is also known to inhibit endothelial cell proliferation and, therefore, is an appropriate anti-angiogenic agent for use in the present method (Heart Development, Harvey & Rosenthal, eds., Academic Press, New York, 1999, see Chapter 14). Anti-angiogenic agents are further discussed in U.S. Pat. No. 5,840,686; International Patent Applications WO 93/24529 and WO 99/04806; Chader, Cell Different., 20, 209-216 (1987); Dawson et al, Science, 285:245-248 (1999); and Browder et al, J. Biol. Chem, 275:1521-1524 (2000).

Numerous cytotoxins and apoptotic factors are known in the art and include, for example, p53, Fas, Fas ligand, Fas-associating protein with death domain (FADD), caspase-3, caspase-8 (FLICE), caspase-10, Apo2L, tumor necrosis factor (TNF)-R1, IκB, ΔIκB, receptor-interacting protein (RIP)-associated ICH-1/CED-3-homologous protein with a death domain (RAIDD), TNF-related apoptosis-inducing ligand (TRAIL), DR4, DR5, a cell death-inducing coding sequence of Bcl-2 which comprises an N-terminal deletion, a cell death-inducing coding sequence of Bcl-x which comprises an N-terminal deletion, Bax, Bak, Bid, Bad, Bik, Bif-2, c-myc, Ras, Raf, PCK kinase, AKT kinase, Akt/PI(3)-kinase, PITSLRE, death-associated protein (DAP) kinase, RIP, JNK/SAPK, Daxx, NIK, MEKK1, ASK1, PKR, and mutants thereof (e.g., dominant negative mutants thereof and dominant positive mutants thereof), and fragments thereof (e.g., active domains thereof), and combinations thereof. Apoptotic, cytotoxic, and cytostatic transcription factors can be used in the present method and include, for example, E2F transcription factors and synthetic cell cycle-independent forms thereof, an AP1 transcription factor, an AP2 transcription factor, an SP transcription factor (e.g., an SP1 transcription factor), a helix-loop-helix transcription factor, a DP transcription factor (e.g., DP1, DP2, and DP3), and mutants thereof (e.g., dominant negative mutants thereof and dominant positive mutants thereof), and fragments thereof (e.g., active domains thereof), and combinations thereof. Apoptotic, cytotoxic, and cytostatic viral proteins include, for example, an adenoviral E1A product, an adenoviral E4/ORF6/7 product, an adenoviral E4/ORF4 product, a cytomegalovirus (CMV) product (e.g., CMV-thymidine kinase (CMV-TK)), a herpes simplex virus (HSV) product (e.g., HSV-TK), a human papillomavirus (HPV) product (e.g., HPVX), and mutants thereof (e.g., dominant negative mutants thereof and dominant positive mutants thereof), and fragments thereof (e.g., active domains thereof), and combinations thereof. Cytotoxins and apoptotic factors are particularly useful in inhibiting cellular proliferation, an important angiogenic process. Other suitable cytotoxins and apoptotic agents can be identified using routine techniques, such as, for instance, cell growth assays and the Tunel assay, respectively.

An anti-sense molecule specific for an angiogenic factor should generally be substantially identical to at least a portion, preferably at least about 20 continuous nucleotides, of the nucleic acid encoding the angiogenic factor to be inhibited, but need not be identical. The anti-sense nucleic acid molecule can be designed such that the inhibitory effect applies to other proteins within a family of genes exhibiting homology or substantial homology to the nucleic acid. The introduced anti-sense nucleic acid molecule also need not be full-length relative to either of the primary transcription product or the fully processed mRNA. Generally, higher homology can be used to compensate for the use of a shorter sequence. Antisense phosphorothioate oligodeoxynucleotides (PS-ODNs) are exemplary of an anti-sense molecule specific for an angiogenic factor.

Ribozymes can be designed that specifically pair with virtually any target RNA and cleave the phosphodiester backbone at a specific location, thereby functionally inactivating the target RNA. In carrying out this cleavage, the ribozyme is not itself altered and is, thus, capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature, 334, 585-591 (1988). Preferably, the ribozyme comprises at least about 20 continuous nucleotides complementary to the target sequence on each side of the active site of the ribozyme.

Examples or soluble receptors include, for example, VEGF-R1 (flt-1), VEGF-R2 (flk/kdr), and VEGF-R3 (fit-4) receptors, as well as VEGF-receptor-chimeric proteins, all of which compete with VEGF receptors on vascular endothelial cells to inhibit endothelial cell growth (Aiello, PNAS, 92:10457 (1995)). Angiogenic factor-specific antibodies and fragments thereof (e.g., Fab, F(ab′)₂, and Fv) that neutralize angiogenic factors or bind receptors for angiogenic factors also can be used in the context of the present inventive method.

Generally, once the substrate has been provided to the test region of interest, whether or not it contains an angiogenesis-modulating factor, it will be allowed to carry out its effects for a period of time. Typically, about 12 hours or more are allowed to pass. Preferably, however, about 18 hours or more are allowed to pass, and, more preferably, about 24 hours are allowed to pass. It will be understood that any suitable time period is available for use in the present invention such that the desired effect of stimulating and/or inhibiting angiogenesis in the test region of interest in achieved.

Whether or not an angiogenesis-modulation factor is included in a substrate provided to a region of interest on the CAM, a test molecule and fluorescent-labeled particle are sequentially administered to a vessel in the CAM after the substrate is allowed to carry out its effects. The test molecule and/or fluorescent-labeled particle is/are desirably administered outside of the region of interest. Moreover, when administering the test molecule and/or fluorescent-labeled particle to a vessel in the CAM, it is preferred that the two molecules are administered to different vessels contained in the CAM.

Prior to administration of the test molecule and/or fluorescent-labeled particle to a vessel in the CAM, it can be desirable to cannulate a vessel such that the test molecule and/or fluorescent-labeled particle can be more easily administered. By “cannulate” is meant providing a tubing device to a vessel such that the test molecule and/or fluorescent-labeled particle can be administered through the tubing device into the vessel. Such tubing i.e., cannulating devices, are known in the art (e.g., a 30-gauge surgical needle). By administering the molecules in this manner, the systemic effects of the test molecule can be measured. Other routes of administration also are available for use in the invention and can include, for example, administration of the molecules by a slow-release patch on the surface of the eggshell, by injection into the yolk sac of the egg, or by topical application on the surface of the CAM.

Typically, the test molecule and/or fluorescent-labeled particle are administered in conjunction with a carrier. Any suitable carrier can be used. Suitable carriers include, for example, DMSO, ethanol, polyethylene glycol (PEG), albumin, or balanced salt solutions, such as sodium chloride, Hank's buffered salt solution, or Ringer's Lactate solution.

The test molecule is administered prior to the administration of the fluorescent-labeled particle. Any suitable test molecule can be used in the context of the invention. Suitable test molecules include, for example, a putative drug, serum from a mammal, or a component of serum from a mammal. When the test molecule is a putative drug, the putative drug can be in the form of a nucleic acid molecule, a protein, polypeptide, or peptide, a small molecule, or an antibody or an antigenically reactive fragment thereof Other test molecules are well-known to those skilled in the art. Accordingly, the test molecules identified herein are in no way limiting.

Any suitable amount of the test molecule can be administered to a vessel in the CAM. For example, the test molecule can be administered in an amount of about 1 μg/egg or more. Preferably, however, the test molecule is administered to a vessel in the CAM in an amount of about 2 μg/egg or more, about 2.5 μg/egg or more, or even about 3 μg/egg or more. More preferably, the test molecule is administered to a vessel in the CAM in an amount of about 3.5 μg/egg or more, about 4 μg/egg or more, or even about 4.5 μg/egg or more. Most preferably, the test molecule is administered to a vessel in the CAM in an amount of about 5 μg/egg or more, about 5.5 μg/egg or more, or even about 6 μg/egg or more. Once administered, the test molecule is allowed to circulate in the vasculature of the fowl egg for a period of time. Preferably, the test molecule is allowed to circulate for about 24 hours or more. More preferably, the test molecule is allowed to circulate for about 36 hours or more, and, most preferably, for about 48 hours or more.

After the test molecule has circulated for a sufficient period of time, the fluorescent-labeled particle is administered to a vessel in the CAM. As with the test molecule, the administration of the fluorescent-labeled particle is typically done in a region outside of the region of interest. Also typically, the fluorescent-labeled particle is administered to a different vessel than the vessel in which the test molecule was administered to.

Any suitable fluorescent-labeled particle can be used in the context of the invention. In that respect, any suitable particle can be associated with any suitable fluorescent moiety (i.e., fluorophore). For example, the particle can comprise a fluorescent-labeled carbohydrate (e.g., dextran), a fluorescent-labeled protein, polypeptide, or peptide (e.g., lectin), or a fluorescent-labeled synthetic polymer (e.g., polystyrene microbeads). Similarly, any suitable fluorescent moiety can be used in the context of the invention. The choice of fluorescent moiety will be determined, at least in part, on which particular imaging device is to be used for image capture. Preferably, the fluorescent-labeled particle is labeled with a fluorescent moiety that can be excited by a laser. In this respect, the fluorescent-labeled particle can be labeled with a fluorescent moiety selected from the group consisting of fluorescein (fluorescein isothiocyanate (FITC)), green fluorescent protein, yellow fluorescent protein, Lucifer yellow, rhodamine, cyanine based compounds, C6-NBD, DIO-Cn-(3), BODIPY-FL, eosin, propidium iodide, Dil-Cn-(3), Cy3, Texas Red, Dil-Cn-(5), allophycocyanin, and Cy5. It will be understood, however, that other fluorescent moieties are suitable for use in the present invention and can be identified by those skilled in the art. Accordingly, the fluorescent moieties identified herein are in no way limiting. Attachment of a particular fluorescent moiety to a particular particle is well-known in the art and described in, for example, Haugland, R P, “Coupling of Monoclonal Antibodies with Fluorophores,” in Methods in Molecular Biology, Vol. 45: Monoclonal Antibody Protocols, W. C. Davis, ed. (Totowa, N.J.: Humana Press, 1995), pp. 205-221.

Any suitable amount of the fluorescent-labeled particle can be administered to a vessel in the CAM. For example, the fluorescent-labeled particle can be administered in an amount of about 30 μl or more. Preferably, however, the fluorescent-labeled particle is administered to a vessel in the CAM in an amount of about 40 μl or more, about 50 μl or more, or even about 60 μl or more. More preferably, the fluorescent-labeled particle is administered to a vessel in the CAM in an amount of about 70 μl or more, about 80 μl or more, or even about 90 μl or more. Most preferably, the fluorescent-labeled particle is administered to a vessel in the CAM in an amount of about 100 μl or more, about 125 μl or more, or even about 150 μl or more. Once administered, the fluorescent-labeled particle is allowed to circulate in the vasculature of the fowl egg for a period of time. Preferably, the fluorescent-labeled particle is allowed to circulate for about 15 minutes or more, for about 20 minutes or more, or even for about 25 minutes or more. Most preferably, the fluorescent-labeled particle is allowed to circulate for about 30 minutes or more in the vasculature of the fowl egg.

After the fluorescent-labeled particle has circulated for a sufficient period of time, the substrate and associated region of interest are removed from the fowl egg. This is accomplished by opening the eggshell by any suitable means to reveal the substrate and associated region of interest. The substrate and region of interest are then removed and a portion is then preserved in a commonly-available fixative, such as formalin or ethanol. Other suitable fixatives will be apparent to those skilled in the art. The substrate and region of interest are then laid out on a glass microscope slide to be analyzed (i.e., to capture an image of the substrate and associated region of interest). When mounting the substrate and region of interest on the slide, it may be desirable to use spacers (e.g., shards of coverslip glass or acrylic support made with nail polish streaks) to separate the substrate and region of interest from the coverslip. Nail polish also can be used to seal the coverslip to the slide.

Any image capture device is suitable for use in the invention. Preferably, when a fluorescent-labeled particle is used, the image capture device is capable of producing a three-dimensional image of the substrate and region of interest. A preferred process involves laser confocal microscopy (LCM). In LCM, a laser light beam is used, through an x-y deflection mechanism, to scan a sample. The mixture of reflected light and emitted fluorescent light is captured and is focused onto a photodetector via a dichroic mirror. The reflected light is deviated by the dichroic mirror, while the emitted fluorescent light passes through the microscope in the direction of the photodetector. Multiple two-dimensional cross-sectional images of the sample are taken along a three-dimensional z-axis. As the laser scans across the sample, the analog light signal, detected by the photodetector, is converted into a digital signal, contributing to a pixel-based image displayed on a computer monitor attached to the LCM. The relative intensity of the fluorescent light, emitted form the laser hit point, corresponds to the intensity of the resulting pixel in the image. A three-dimensional reconstruction of a substrate and region of interest can be generated by stacking the two-dimensional cross-sectional images collected in series. LCM is generally described in, for example, Cheng et al., “Multidimensional Microscopy.” Springer, Verlag, N.Y. (1994), Cogswell et al., “Three-dimensional Microscopy:lmage Acquisition and Processing.” SPIE, Bellingham, Wash. (1994), Matsumoto, B., “Cell Biological Applications of Confocal Microscopy.” Academic Press, San Diego, Calif. (1993), Pawley, J. B., “Handbook of Confocal Microscopy.” Plenum, N.Y. (1990), and Stevens et al., “Three-Dimensional Confocal Microscopy:Volume Investigation of Biological Systems.” Academic Press, London (1994).

In performing image capture using a light confocal microscope, various steps can be taken to improve the quality of the image. For example, fading and/or bleaching can occur. Several theories have been proposed as to the cause of fading and/or bleaching, including the damaging effects of oxygen free-radicals and protein denaturation. In this respect, antifade reagents can be utilized to slow this fading process, allowing longer observation times, fluorometry, and pattern recognition, if desired. Such antifade reagents are well-known in the art and are described in, for example, Krenik et al., J. Immunol. Methods, 117:91-97(1989).

Another factor in data acquisition using a LCM is the signal-to-noise ratio (SNR) in the optical sections of the substrate and region of interest. In this respect, the adjustment of the detector pinhole is of critical importance. For example if the SNR is too low, the diameter of the pinhole can be increased, thereby allowing increased detection of signal at the expense of resolution. However, if the pinhole is made too large, both the SNR and resolution will decrease. Conversely, if the pinhole is too small, the signal can be lost.

Misalignments can usually be corrected using computer software after the stacks have been collected, and can be in the form of translations (shifts in the X and/or Y plane) or rotations in the X and Y plane. While the latter is more complex to correct and can introduce sampling variations in the data, correction of both forms of misalignment have the effect of reducing the bounding area of the original X and Y image size that contributes to the three-dimensional volume. Some newer confocal microscopes have automated methods of changing filters which greatly reduces or eliminates alignment problems for multilabel imaging.

After an image stack has been acquired, it may be preprocessed to improve image quality prior to three-dimensional reconstruction. The preprocessing typically involves application of image filters (mathematical algorithms implemented in software) to the entire data set to remove noise and artifacts, smooth or sharpen the images, or to correct for problems with contrast and/or brightness. While these filters are generally performed as preprocessing steps, they also can be carried out after a three-dimensional model has been reconstructed from the image stack. Median and Gaussian filters have the general affect of smoothing images. These are used to eliminate noise and background artifacts and to smooth sharp edges, but also tend to remove some of the detail in small objects.

Sharpening filters can be used to emphasize details in the image stack, but also have the effect of highlighting noise and other small artifacts. The application of sharpening filters is most useful when the image stack consists of fine structural components of a specimen, or when edge enhancement is desired.

It is important to realize that the application of filters to the data set can ultimately affect quantitative measurements of three-dimensional reconstructions produced from it. As such, the application of filters in some instances is only used for display purposes, and quantitative measurements are made on the unprocessed data.

The contrast and brightness of the image stack can be adjusted to enhance perception of the sampled specimen. This is usually done by changing the ramping of the grey scale values for the dataset. Histogram equalization can be used to improve contrast by a non-linear mapping of the grey levels in an image. This technique is most commonly used when the grey levels are concentrated in a small portion of the range of possible values.

Segmentation refers to the process of extracting the desired object (or objects) of interest from the background in an image or data volume. There are a variety of techniques that are used to do this, ranging from the simple (such as thresholding and masking) to the. complex (such as edge/boundary detection, region growing and clustering algorithms). Segmentation can be aided through manual intervention or handled automatically through software algorithms. It can be performed before building the three-dimensional reconstruction by processing of images in the image stack, or after the three-dimensional model has been formed. Typically, thresholding and/or masking is employed in the present inventive method.

Thresholding involves limiting the intensity values within an individual image or the entire image stack to a certain bounded range (or ranges). For example, since each pixel in an 8-bit confocal image (with values 0 to 255) corresponds to fluorescence intensity at a point within the substrate and region of interest, the pixels with lower values represent areas with lower fluorescence, while the pixels with higher values represent brighter regions. It can be decided that all pixels below a certain value do not contribute significantly to the object(s) of interest and hence can be eliminated. This can be done by scanning the image(s) one pixel at a time, and keeping that pixel if it is above the selected intensity value, or setting it to 0 if it is below that value. In a similar manner, thresholding also can be used to eliminate non-consecutive ranges of intensities, while preserving the regions containing the intensities of interest.

Masking is a procedure whereby an enclosed region(s) of an image (or of the image stack) is/are defined for processing. This can be done either by manually tracing around the regions of interest (e.g., with a mouse in a graphics application) or by an automated routine. An application of this is to use a two-dimensional stacked projection of an image to define the image mask. The stacked projection of the image stack is a single image that represents the sum of all of the images in the image stack (these images usually can be provided automatically from software supplied with the LCM). If the object of interest has a closed, continuous surface (such as that of a neuron), the stacked projection defines the absolute boundaries of the object in two-dimensions. A mask can be formed by either manually tracing around the boundaries of the object(s) of interest in the stacked projection or by absolute thresholding (making all intensities above a certain value white and all below this value black). The mask then can be applied to the entire image stack, such that regions falling within the mask selection area are preserved, whereas areas outside this region are eliminated (e.g. set to 0). After the mask has been applied, thresholding and image filtering methods can be used to aid in removing the remaining undesired regions.

Images that are obtained from the LCM are typically in 8-bit gray scale format. Color can be added to these images prior to three-dimensional reconstruction to highlight regions of interest or to differentiate selectively between different parts of an object. The most common method of assigning color to intensity images is by thresholding. Various intensity ranges are assigned different colors, with the color in each range usually being ramped from dark to light to reflect fluorescence intensity.

After the image stack is processed by two-dimensional image processing techniques, it then can be reconstructed into a three-dimensional volumetric dataset. This is usually achieved using either volume- or surface-rendering techniques.

Volume-rendering is a computer graphics technique whereby the object or phenomenon of interest is sampled or subdivided into many cubic building blocks called voxels (or volume elements). A voxel is the three-dimensional counterpart of the two-dimensional pixel and is a measure of unit volume. Each voxel carries one or more values for some measured or calculated property of the volume (such as intensity values in the case of LCM data) and is typically represented by a unit cube. The three-dimensional voxel sets are assembled from multiple two-dimensional images (such as the LCM image stack), and are displayed by projecting these images into two-dimensional pixel space, where they are stored in a frame buffer. Volumes rendered in this manner have been likened to a translucent suspension of particles in three-dimensional space.

In surface-rendering the volumetric data must first be converted into geometric primitives, by a process such as isosurfacing, isocontouring, surface extraction, or border following. These primitives (such as polygon meshes or contours) are then rendered for display using conventional geometric rendering techniques.

Both techniques have advantages and disadvantages. A major advantage of the volume-rendering technique is that the three-dimensional volume can be displayed without any knowledge of the geometry of the dataset and, hence, without intermediate conversion to a surface representation. This conversion step in surface-rendering sometimes can be quite complex, especially if surfaces are not well-defined (i.e. noisy two-dimensional images) and can require a lot of user intervention (such as manual contour-tracing). On the other hand, because the three-dimensional dataset is reduced to a set of geometric primitives in surface-rendering, this can result in a significant reduction in the amount of data to be stored, and can provide fast display and manipulation of the three-dimensional reconstructions produced by this method. By contrast, since all of the image stack data is used for volume-rendering, computers with lots of memory and processing power are required to handle volumes rendered in this manner. Because the entire dataset is preserved in volume-rendering, any part, including internal structures and details (which may be lost when reducing to geometric structures with surface-rendering) can be viewed.

In the present inventive method, the LCM is directly linked to a computer, such that the data from the LCM is sent directly to the computer for analysis. The computer analysis typically involves the analysis of the relative fluorescent brightness of each pixel contained in three-dimensional image to determine an FVD value for each vessel contained in each cross-sectional image. These FVD values for each vessel contained in each cross-sectional image then can be used to determine an FVD value for the region of interest. For example, the FVD value of the region of interest can be determined by (a) assigning a brightness value ranging from 0 to 255 to each pixel, (b) applying a thresholding algorithm to the image, (c) selecting a low threshold such that any pixel with less brightness than the low threshold is deleted, (d) optionally, selecting a high threshold such that any pixel with greater brightness than the high threshold is deleted, (e) summing the remaining pixels to determine the FVD value of all of the vessels in each cross-sectional image of the region of interest, and (f) averaging the FVD values of at least two of the brightest cross-sectional images to determine the FVD value of the region of interest. Although current software allows for a density measurement, it also is contemplated that additional software can be developed to measure the angiogenic or antiangiogenic activity of a test molecule by other means. For example, it is contemplated that software can be developed to calculate the number of vessels in the substrate and region of interest that is scanned by LCM.

When an agent to measure metabolic activity is used in the method, spectrophotometry is desirably used to determine absorbance values of the test region of interest and the control region of interest.

### EXAMPLES

The following examples further illustrate the invention but, of course, should not be construed as in any way limiting its scope.

### Example 1

This example demonstrates the effect that an antiangiogenic agent has on the FVD value of the chorioallantoic membrane tissue of an embryonated chicken egg.

Eighteen 10-day-old embryonated chicken eggs were obtained. A window was made in the shell of each egg. The chorioallantoic membranes were dropped to allow placement of a filter paper disc saturated with either basic fibroblast growth factor (bFGF) (1 μg/ml), as an angiogenic stimulator, or with a control solution (Clonetics EBM-2 media, Bio-Whittaker, Walersville, Md.). Twenty-four hours were allowed to elapse and a vessel was cannulated in each egg. Embryos that had received a filter disc saturated with bFGF then received a systemic injection of either Fumagillin (5 μg/egg, Calbiochem, San Diego, Calif.) in 0.1% dimethyl sulfoxide (DMSO) (Group A, n=3), or an injection of 0.1% DMSO carrier alone (Group B, n=5). Fumagillin is an organic compound with established antiangiogenic effects (see, e.g., Ingber et al., Nature, 348:555-557 (1990)). Embryos that had received a filter disc saturated with the control molecule received 0.1% DMSO carrier injected systemically (Group C, n=10).

Forty-eight hours were allowed to elapse, and all eggs then received a systemic injection of fluorescein isothiocyanate (FITC)-Dextran (1 mg/ml), which was allowed to circulate for 30 minutes. Filter discs and associated CAM tissue (i.e., the region of interest) were excised from the egg and preserved in 10% formalin. The filter disc and region of interest were then laid out on a glass microscope slide and viewed using a laser confocal microscope (LSM 510 Laser Scanning Microscope, Carl Zeiss, Inc., Thronwood, N.Y.). The filter disc and region of interest were stimulated with an argon laser emitting at a wavelength of 488 nm. The resultant excitation was read using fluorescence filters at wavelengths of 505 nm and greater. The image was captured using LSM 5 Pascal software (Carl Zeiss, Inc.), through a microscope objective of 1.25×. The capture image data was formatted as 1024 pixels wide by 1024 pixels long, resulting in an overall image of 1,048,576 pixels, each with a brightness measured on a scale from 0 to 255. Multiple cross-sectional images were taken along a three-dimensional z-axis at 200 micron intervals through each filter disc and region of interest from each egg. Each filter disc and region of interest was thereby determined to have a fluorescent vascular density reflective of the density of vasculature of the CAM tissue associated with the filter disc. The results are set forth below in Table 1. One filter disc in Group C was eliminated from analysis owing to gross extravasation of fluorescein, rendering it unreadable (represented by “−” in Table 1).

TABLE 1

| | Treatment | Treatment | Treatment |
|---|---|---|---|
| | Group A | Group B | Group C |
| FVD | 0.0673 | 2.5401 | 0.2501 |
| | 0.0533 | 3.7715 | 0.1207 |
| | 1.5029 | 1.9767 | 0.0007 |
| | | 0.6873 | 0.1919 |
| | | 2.6344 | 0.0268 |
| | | | 0.0001 |
| | | | 0.4376 |
| | | | 2.7498 |
| | | | 2.1193 |
| | | | — |
| Group Mean | 0.5412 | 2.322 | 0.6552 |

As indicated by the results set forth above, systemic injection of Fumagillin, an antiangiogenic agent, is shown on average to reduce the fluorescent vascular density of bFGF-stimulated specimens (Group A), relative to those embryos that received bFGF stimulation and then injections of a control molecule (Group B). The fluorescent vascular density of Fumagillin-treated specimens (Group A) is comparable to that of specimens that received no stimulation of angiogenesis at all (Group C).

As described above, a modified version of the CAM assay comprising systemic injection of FITC-dextran created a quantitative measurement of vascular density. Such an assay can aid in the investigation of molecular mechanisms underlying the angiogenic response following exposure to agents, the identification of novel targets, and the design of novel therapeutics.

### Example 2

This example demonstrates the direct quantitative measurement of endothelial cell proliferation in the CAM assay.

Ten 10-day old eggs were divided into four groups and prepared as in Example 1. The eggs received (A) filter disks saturated with a vehicle solution (0.1% bovine serum albumin (BSA)/phosphate-buffered saline (PBS)) and systemic injection of a carrier solution (0.1% DMSO), (B) filter disks saturated with a stimulator solution (2.0 μg/mL FGF) and systemic injection of carrier solution (0.1% DMSO), (C) filter disks saturated with a stimulator solution (2.0 μg/mL FGF) and systemic injection of an inhibitor (100 μM Fumagillin), or (D) filter disks saturated with a stimulator solution (2.0 μg/mL FGF) and systemic injection of an inhibitor (3 mg/mL LM609). Twenty-four hours after systemic injection of the inhibitor (Fumagillin or LM609) or carrier (0.1% DMSO), Day 12 eggs from Groups A-D were removed from the incubator and underwent a cell proliferation assay via an XTT cell proliferation kit (Roche Applied Science).

XTT is a tetrazolium salt, which is reduced within the mitochondria of metabolically active cells to form a colored formazan dye. The amount of formazan dye produced is quantitatively measured by spectrophotometrical absorbance (e.g., by an ELISA plate reader) at 450-500 nm. Similar reagents that can be used to measure metabolic activity include MTT (550-600 nm) and WST-1 (420-480 nm) (Roche Applied Science).

5 mL of XTT labeling reagent and 0.1 mL electron coupling reagent were mixed together and 30 μL of the mixture was applied onto the CAM disk via pipette. A small piece of sterile Scotch tape was applied over the central window and the eggs were returned to the incubator for 24 hours. At Day 13, the disks were harvested for light photomicroscopy and the mean spectrophotometrical absorbance at 450 nm was measured (reference wavelength 620 nm (Titertek Multiskan Ascent Plate Reader)).

A significant difference was observed between the mean spectrophotometrical absorbance of stimulated CAM disks and unstimulated disks (p=0.028, student's T test) (Groups B and A, respectively). Additionally, significant differences were observed between stimulated disks and CAM disks treated with systemic injection of Fumagillin (p=0.018, student's T test) and LM609 (Med Immune, Gaithersburg, Md.) (p=0.029, student's T test) (Groups B, C, and D, respectively). As similarly observed in Example 1, systemic injection of Fumagillin or LM609 reduced the spectrophotometrical absorbance of the FGF-stimulated specimens relative to those embryos that received FGF stimulation and then injections of the carrier molecule (0.1% DMSO), and the spectrophotometrical absorbance values of Fumagillin- and LM609-treated specimens were comparable to those of specimens that received no stimulation of angiogenesis at all.

The XTT assay is a useful method to quantify endothelial cell proliferation or inhibition in the CAM assay. This CAM-XTT assay can be used as a complementary method to the modified CAM assay utilizing systemic injection of FITC-dextran.

### Comparative Example 1

This example demonstrates the benefit of using the method of the present invention as compared to one of the prior art.

Filter discs and associated CAM tissue were prepared as described in Example 1. However, instead of using laser confocal microscopy to determine an FVD value for a test region of interest, the filter discs and associated CAM tissue were photographed under light microscopy. The light microscopic photographs were evaluated independently by three blinded observers, all experts in the study of angiogenesis. The filter discs and associated CAM tissues were scored as follows:
1+=inhibition of angiogenesis (i.e., the least angiogenesis)
2+=intermediate amount of angiogenesis
3+=stimulation of angiogenesis (i.e., the most angiogenesis)

The scores, ranging from 1 to 3 for each disc, were then averaged for each treatment Group. Results are set forth in Tables 2-4 below with each table representing the scores given by one of the blinded observers. If the blinded observer felt that a disc had too much hemorrhage or artifact to make an accurate assessment of angiogenesis, it was eliminated from that observer's assessment (represented by a “−” in Tables 2-4).

TABLE 2

| | Treatment | Treatment | Treatment |
|---|---|---|---|
| | Group A | Group B | Group C |
| Score | 2 | 2 | 3 |
| | 2 | 3 | 2 |
| | 2 | 1 | 2 |
| | | 2 | 1 |
| | | 1 | 1 |
| | | | 1 |
| | | | 1 |
| | | | 1 |
| | | | 2 |
| | | | 3 |
| Group Mean | 2 | 1.8 | 1.7 |

TABLE 3

| | Treatment | Treatment | Treatment |
|---|---|---|---|
| | Group A | Group B | Group C |
| Score | 2 | 2 | 3 |
| | 3 | 3 | 2 |
| | 3 | 1 | — |
| | | 3 | 2 |
| | | 3 | 3 |
| | | | 1 |
| | | | 2 |
| | | | 2 |
| | | | 2 |
| | | | 3 |
| Group Mean | 2.7 | 2.4 | 2.2 |

TABLE 4

| | Treatment | Treatment | Treatment |
|---|---|---|---|
| | Group A | Group B | Group C |
| Score | 3 | 2 | 3 |
| | 2 | 3 | 2 |
| | 1 | 1 | 3 |
| | | 3 | 1 |
| | | 2 | 2 |
| | | | 1 |
| | | | 1 |
| | | | 1 |
| | | | 2 |
| | | | 3 |
| Group Mean | 2 | 2.2 | 1.9 |

As indicated by the results set forth in Tables 2-4, when the filter discs were evaluated by blinded observers using a detection method of the prior art, none of the three independent, blinded observers could visually appreciate differences between the groups when the methodology of blinded grading was applied. However, as indicated in Example 1, differences were seen when an FVD value was assigned to each group. Accordingly, the present inventive method shows superiority to previous detection methods in the art.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-25. (canceled)

26. A method of measuring the angiogenic or antiangiogenic activity of a test molecule comprising:
(a) obtaining an embryonated fowl egg,
(b) creating a window in the shell of the fowl egg, such that the chorioallantoic membrane (CAM) is exposed,
(c) providing to a test region of interest on the CAM a substrate,
(d) administering to a vessel located in the CAM a test molecule,
(e) administering to a vessel located in the CAM a fluorescent-labeled particle, such that the fluorescent-labeled particle travels through each vessel contained in the test region of interest,
(f) removing the substrate and the test region of interest from the fowl egg,
(g) capturing a three-dimensional image of the test region of interest, wherein the three-dimensional image comprises a plurality of pixels, such that a fluorescent vascular density (FVD) value can be assigned to the test region of interest, and
(h) comparing the FVD value of the test region of interest with the FVD value of a control region of interest that was prepared in the same manner as the test region of interest but without the administration of a test molecule or with the administration of a control molecule, such that the angiogenic or antiangiogenic activity of the test molecule is measured,
wherein a lower FVD value of the test region of interest as compared to the FVD value of the control region of interest is indicative of the test molecule being useful as an inhibitor of angiogenesis, and
wherein a higher FVD value of the test region of interest as compared to the FVD value of the control region of interest is indicative of the test molecule being useful as a stimulator of angiogenesis.

27. The method of claim 26, wherein the embryonated fowl egg is a chicken egg.

28. The method of claim 26, wherein the substrate is selected from the group consisting of glass, plastic, nylon, silicon, polytetrafluroethylene, Matrigel, collagen, fibrinogen, agarose, methylcellulose, and filter paper.

29. The method of claim 28, wherein the substrate comprises a stimulator of angiogenesis, such that angiogenesis is stimulated in the region of interest.

30. The method of claim 29, wherein the stimulator of angiogenesis is selected from the group consisting of a synthetic molecule, a nucleic acid sequence encoding a stimulator of angiogenesis, a polypeptide that can stimulate angiogenesis, a biological tissue containing a stimulator of angiogenesis, and a cell containing a stimulator of angiogenesis.

31. The method of claim 28, wherein the substrate comprises an inhibitor of angiogenesis, such that angiogenesis is inhibited in the region of interest.

32. The method of claim 31, wherein the inhibitor of angiogenesis is selected from the group consisting of a synthetic molecule, a nucleic acid sequence encoding an inhibitor of angiogenesis, a polypeptide that can inhibit angiogenesis, a biological tissue containing an inhibitor of angiogenesis, and a cell containing an inhibitor of angiogenesis.

33. The method of claim 26, wherein the test molecule and the fluorescent-labeled particle are administered to different vessels in the CAM.

34. The method of claim 33, wherein each vessel is cannulated prior to administration of the test molecule and the fluorescent-labeled particle.

35. The method of claim 34, wherein the fluorescent-labeled particle is selected from the group consisting of a fluorescent-labeled carbohydrate, a fluorescent-labeled protein, polypeptide, or peptide, and a fluorescent-labeled synthetic polymer.

36. The method of claim 35, wherein the fluorescent-labeled particle is labeled with a fluorescent moiety selected from the group consisting of fluorescein, green fluorescent protein, yellow fluorescent protein, Lucifer yellow, rhodamine, cyanine based compounds, C6-NBD, DIO-Cn-(3), BODIPY-FL, eosin, propidium iodide, Dil-Cn-(3), Cy3, Texas Red, Dil-Cn-(5), allophycocyanin, and Cy5.

37. The method of claim 36, wherein the fluorescent-labeled particle is labeled with a fluorescent moiety that can be excited by a laser and the three-dimensional image is captured by laser confocal microscopy.

38. The method of claim 37, wherein the three-dimensional image is formed by taking multiple cross-sectional images along a three-dimensional z-axis of the region of interest.

39. The method of claim 38, wherein the three-dimensional image is analyzed by computer analysis of the relative fluorescent brightness of each pixel contained in the three-dimensional image to determine an FVD value for each vessel contained in each cross-sectional image.

40. The method of claim 39, wherein the FVD value of the region of interest is calculated by:
(a) assigning a brightness value ranging from 0 to 255 to each pixel,
(b) applying a thresholding algorithm to the image,
(c) selecting a low threshold such that any pixel with less brightness than the low threshold is deleted,
(d) optionally, selecting a high threshold such that any pixel with greater brightness than the high threshold is deleted,
(e) summing the remaining pixels to determine the FVD value of all of the vessels in each cross-sectional image of the region of interest, and
(f) averaging the FVD values of at least two of the brightest cross-sectional images to determine the FVD value of the region of interest.

41. A method of measuring the angiogenic or antiangiogenic activity of a test molecule comprising:
(a) obtaining an embryonated fowl egg,
(b) creating a window in the shell of the fowl egg, such that the CAM is exposed,
(c) providing to a test region of interest on the CAM a substrate,
(d) administering to a vessel located in the CAM a test molecule,
(e) administering to a vessel located in the CAM an agent to measure metabolic activity,
(f) removing the substrate and the test region of interest from the fowl egg,
(g) measuring the spectrophotometric absorbance value of the test region of interest, and
(h) comparing the spectrophotometric absorbance value of the test region of interest with the spectrophotometric absorbance value of a control region of interest that was prepared in the same manner as the test region of interest but without the administration of a test molecule or with the administration of a control molecule, such that the angiogenic or antiangiogenic activity of the test molecule is measured,
wherein a lower spectrophotometric absorbance value of the test region of interest as compared to the spectrophotometric absorbance value of the control region of interest is indicative of the test molecule being useful as an inhibitor of angiogenesis, and
wherein a higher spectrophotometric absorbance value of the test region of interest as compared to the spectrophotometric absorbance value of the control region of interest is indicative of the test molecule being useful as a stimulator of angiogenesis.

42. The method of claim 41, wherein the agent is XTT, MTT, or WST-1.

43. The method of claim 41, wherein the embryonated fowl egg is a chicken egg.

44. The method of claim 42, wherein the substrate is selected from the group consisting of glass, plastic, nylon, silicon, polytetrafluroethylene, Matrigel, collagen, fibrinogen, agarose, methylcellulose, and filter paper.

45. The method of claim 44, wherein the substrate comprises a stimulator of angiogenesis, such that angiogenesis is stimulated in the region of interest.

46. The method of claim 44, wherein the stimulator of angiogenesis is selected from the group consisting of a synthetic molecule, a nucleic acid sequence encoding a stimulator of angiogenesis, a polypeptide that can stimulate angiogenesis, a biological tissue containing a stimulator of angiogenesis, and a cell containing a stimulator of angiogenesis.

47. The method of claim 44, wherein the substrate comprises an inhibitor of angiogenesis, such that angiogenesis is inhibited in the region of interest.

48. The method of claim 47, wherein the inhibitor of angiogenesis is selected from the group consisting of a synthetic molecule, a nucleic acid sequence encoding an inhibitor of angiogenesis, a polypeptide that can inhibit angiogenesis, a biological tissue containing an inhibitor of angiogenesis, and a cell containing an inhibitor of angiogenesis.

49. The method of claim 46, wherein the test molecule and the agent to measure metabolic activity are administered to different vessels in the CAM.

50. The method of claim 49, wherein each vessel is cannulated prior to administration of the test molecule and the agent to measure metabolic activity.
